(19) United States
(12) Patent Application Publication
(10) Pub. No.: US 2006/0149053 A1
(43) Pub. Date: Jul. 6, 2006

(54) Dextrinized, saccharide-derivatized oligosaccharides

(75) Inventors: Richard L. Antrim, Solon, IA (US); Frank W. Barresi, Coralville, IA (US); Roger E. McPherson, Muscatine, IA (US)

Correspondence Address: BANNER & WITCOFF, LTD., TEN SOUTH WACKER DRIVE, SUITE 3000, CHICAGO, IL, 60606, US

(73) Assignee: Grain Processing Corporation, Muscatine, IA (US)

(21) Appl. No.: 11/292,246

(22) Filed: Dec. 1, 2005

Related U.S. Application Data
(63) Continuation-in-part of application No. 10/874,686, filed on Jun. 22, 2004.
(63) Continuation-in-part of application No. 10/601,912, filed on Jun. 23, 2003.
(60) Provisional application No. 60/482,045, filed on Jun. 23, 2003.
(60) Provisional application No. 60/390,570, filed on Jun. 21, 2002.

(51) Int. Cl. C12P 19/04; C08B 37/00

(52) U.S. Cl. 536/123

(57) ABSTRACT

Saccharide-derivatized oligosaccharides prepared by extruding a reaction mixture comprising a saccharide having a degree of polymerization ranging from 1 to 4 and a starch having a degree of polymerization of at least 200, wherein the extruding imparts sufficient energy and work to derivatize the starch with the saccharide.

This application is a continuation-in-part of U.S. Ser. No. 10/874,686, filed Jun. 22, 2004 which claims priority from U.S. Provisional Application No. 60/482,045, filed Jun. 23, 2003. This application is also a continuation-in-part of U.S. Ser. No 10/601,912, filed Jun. 23, 2003, which claims priority from U.S. Provisional Application No. 60/390,570, filed Jun. 21, 2002. The entire disclosures of each of the foregoing applications are hereby incorporated by reference.

### FIELD OF INVENTION

The invention is in the field of starch and starch derivatives. More particularly, the invention is directed towards an oligosaccharide compound and composition that are useful as low-calorie bulking agents and slow energy release products.

### BACKGROUND OF THE INVENTION

Many substances are used in the manufacture of foods: intended for persons and animals who must restrict their intake of carbohydrates or calories. Such substances generally should be of low caloric value and of a generally non-nutritive nature. In addition, such substances must not be toxic or unwholesome. The foods or animal feeds produced using such substances preferably are formulated such that they resemble higher calorie products in texture, taste and physical appearance.

Among such substances are synthetic sweeteners. When a synthetic sweetener such as saccharin or aspartame is used in a dietetic food as a substitute for sugar, the other physical properties which would have been imparted by sugar, such as appearance, bulk mass, and texture, must also be imparted to the dietetic food by a separate ingredient. For instance, saccharin and aspartame both are substantially sweeter than sugar. It is often necessary to provide a low-calorie, non-nutritive carrier so that the bulk mass, appearance, and texture of the added sweetener approximates that of sugar.

The prior art has provided numerous such bulking agents. One such bulking agent that is well known in literature is polydextrose, as is taught, for instance, in U.S. Pat. Nos. 3,766,165 and 3,876,794 (both to Rennhard). Polydextrose is a product of melt polymerization of glucose or maltose, generally using edible acids, such as citric acid, as catalysts and cross-linking agents. Polydextrose has a substantially reduced caloric value relative to sugar (about 1 Kcal/gm), or about 25% that of dextrose. As such, polydextrose may be used as a bulking agent in connection with synthetic sweeteners and other applications.

Although polydextrose is satisfactory for many purposes as a non-nutritive bulking agent, there exist several practical difficulties concerning the use of this material. For instance, the production of polydextrose is not without difficulty. Polydextrose generally is prepared in a condensation reaction that is performed under harsh conditions. As such, the condensation reaction often results in a dark colored product that has an undesirable acidic and bitter flavor. Numerous efforts have been made to address this problem. For instance, efforts to improve on the manufacturing process of polydextrose have been provided. As taught, for instance, in EP 404,227 (to Coöperatieve Vereniging Suiker Unie V.A.) and in U.S. Pat. No. 5,015,500 (to Elmore), various extrusion techniques for polydextrose have been taught. Another reference, U.S. Pat. No. 5,558,899 (to Kuzee et al.), purports to disclose the production of polydextrose via use of microwave energy. Other references purport to disclose methods to improve the taste or flavor of polydextrose. For instance, U.S. Pat. No. 4,622,233 (to Torres) purportedly teaches peroxide bleaching of polydextrose in an alcohol solvent. U.S. Pat. No. 4,948,596 (to Bunich et al.) purportedly discloses a liquid/liquid extraction process for purifying polydextrose. U.S. Pat. No. 4,956,458 (to Luo et al.) is said to disclose another process said to be useful for purifying polydextrose. U.S. Pat. No. 5,091,015 (to Bunich); U.S. Pat. No. 5,677,593 (to Guzek et al.); and U.S. Pat. No. 5,831,082 (to An et al.) purport to teach chromatographic methods for purifying polydextrose. U.S. Pat. No. 5,573,794 (to Duflot) purports to disclose glucose oxidase treatment of polydextrose followed by ion exchange chromatography. Finally, U.S. Pat. No. 5,601,863 (to Borden et al.) and U.S. Pat. No. 5,424,418 (to Duflot et al.) disclose hydrogenated polydextrose.

All of the foregoing approaches to polydextrose production are somewhat limited in utility. One principal drawback common to all of these approaches is that the polydextrose produced by any process typically includes substantial quantities of undesired color and flavor components, and substantial effort is required to reduce the levels of such components to acceptable levels. Moreover, the polydextrose product that is obtained in a typical condensation reaction has a low molecular weight. It would be desirable to have a low calorie bulking agent that has the properties of a higher molecular weight product such as a maltodextrin. More recently, to address this latter concern, a number of patents, including U.S. Pat. No. 5,264,568 (to Yamada et al.); U.S. Pat. No. 5,358,729; 5,364,652; and U.S. Pat. No. 5,430,141 (all to Ohkuma et al.); and EP 368,451 (to Matsutani Chemical Industries Co. Ltd.) purport to disclose a product, commonly known as FIBERSOL, that is formed by starch pyrodextrinization followed by enzymatic hydrolysis to leave an undigestive carbohydrate remnant. It is said that the disclosed product can be hydrogenated and/or ion exchanged to give a final product with reduced calorie content and soluble fiber benefits. This product is higher is molecular weight than most polydextroses, and therefore has properties that rival maltodextrins. However, the product also suffers from low processing yields, significant processing complexities, and high final cost.

In addition to such low- or non-caloric products, there is a demand for a carbohydrate product that can be digested slowly. Ideally, the carbohydrate product should be fully digestible, yet should deliver calories evenly for an extended period of time. Typically, carbohydrates that are fully digestible are digested rapidly, causing a spike in blood glucose levels soon after ingestion (a hyperglycemic state) followed by a drop in blood glucose level (a hypoglycemic state) due to over-expression of insulin. For some people, potential ill effects such as increase risk of cardiovascular disease and hypoglycemic related side effects such as blurred vision, loss of consciousness, and diminished mental acuity can result from such fluctuation in blood glucose levels.

The prior art has provided numerous controlled energy released products. Hydrogenated starch hydrolysates such as LYCASIN® (Roquette Freres) and HYSTAR® (SPI Polyols) are examples of such products. It is known that these products are digested more slowly then their non-hydrogenated counterparts, because the digestion products of a hydrogenated starch hydrolyzate are glucose and sorbitol, and the sorbitol component of the mixture is digested more slowly than glucose. See Dwivedi, Food Science & Technology Books, Vol. 17 pp. 165-183 (1986). One drawback of hydrogenated starch hydrolysates is that they have relatively high osmolality and are associated with high level of sorbitol and maltitol digestion products that can cause cramping and diarrhea.

Another document, International Publication WO 96/31129, discloses a mixture of rapidly digestible, slowly digestible, and non-digestible products. For instance, this document teaches that a combination of rapidly digested carbohydrate with a slowly digested complex carbohydrate such as raw cornstarch in conjunction with proteins and fats can be used in the control of blood glucose levels. The slowly digested product is a raw starch, which is not fully digested and, because of its lack of cold water solubility, is only amenable to use in solid products.

Chemically modified starches, such as oxidized, dextrinized, and etherified starches also have been examined as candidates for controlled energy release (see, e.g., J. Agric. Food Chem. 47:4178 (1999)). In general, it has been found that the more chemically modified a material is, the less digestible the material is. Most of these products tend to have no digestibility or very low digestibility, and thus may be considered to be resistant starches or soluble fiber.

It is a general object of the present invention to provide an oligosaccharide product. In some preferred embodiments, it is an object to provide a low-calorie oligosaccharide product that does not suffer from the same disadvantages as polydextrose and that can be produced more readily and inexpensively than the enzymatically treated starch pyrodextrinization product hereinbefore described. In other embodiments, it is a general object to provide an oligosaccharides product that releases nutritional energy slowly in comparison to glucose.

### SUMMARY OF THE INVENTION

It has now been found that dextrinized oligosaccharides may be prepared from starch. Surprisingly, such products exhibit improved color over products produced from oligosaccharides (maltodextrins, for example). Like dextrinized oligosaccharide products produced from maltodextrins, dextrinized oligosaccharides prepared from starch can function as bulking agents or as slow energy release compounds.

Generally, in accordance with the claimed invention, a starch, such as corn starch, is dextrinized and derivatized by extrusion in the presence of a saccharide have a degree of polymerization of 1 to 4, such as dextrose. The starch should have a degree of polymerization of at least 200, and preferably at least 500. Mixtures of one starch with other starches, likewise may be dextrinized and derivatized. Upon extrusion, an oligosaccharide product will be produced.

The oligosaccharide and the process for its preparation offer a number of unexpected properties and advantages not heretofore realized. For instance, in some embodiments, the product has low digestibility, and thus is suitable in a number of applications as a bulking agent, a product carrier, or the like. In other embodiments, the product can be made to release nutritional energy slowly relative to glucose. The product does not require large amounts of acid for catalysis, and in some instances, the product may be prepared with no acid catalysis whatsoever. The product can be made to have a higher molecular weight than most commercially available polydextrose products, thus making the product similar in properties to many maltodextrins and therefore suitable for use in more applications than is polydextrose. Finally, and perhaps most surprisingly, color components and undesired flavor components formed in the process readily can be kept to a minimum, and these undesired components readily can be removed. Not as much polymerization is required for production of the product as is required in the preparation of polydextrose, and thus the harsh reaction conditions typically required for polydextrose production are not required. The preferred process for production of the derivatized product is simple, with a high tolerance for moisture content in the starting materials. Thus, in preferred embodiments, there is no need to take expensive steps to avoid moisture uptake in the starting materials.

It has been found in preferred embodiments that the solubility of the saccharide-derivatized oligosaccharides is greater than 90% at 25° C., and that the Minolta L color of the saccharide-derivatized oligosaccharides is greater than 85 even prior to a decolorization step.

Also provided by the invention are a process for preparing an oligosaccharide product and a process for preparing a mixture of oligosaccharides, both as set forth hereinbelow.

### DETAILED DESCRIPTION OF THE PREFERRED EMBODIMENTS

The present invention provides a mixture of oligosaccharides prepared by extruding a reaction mixture that includes a saccharide having a degree of polymerization ranging from 1 to 4, and a starch having a degree of polymerization of at least 200, wherein the extruding imparts sufficient energy and force to derivatize starting with the saccharide. The resulting product is an oligosaccharide in the form of a dextrin.

The present invention further provides a process for preparing a mixture of oligosaccharides, comprising providing a reaction mixture comprising a saccharide having a degree of polymerization ranging from 1 to 4, and a starch having a degree of polymerization of at least 200; selecting a desired polymolecularity index for the mixture of oligosaccharides; selecting extrusion conditions, which, when applied, produce the polymolecularity index; and extruding the reaction mixture under the extrusion conditions, wherein the extruding imparts sufficient energy and work to derivatize the starch with the saccharide to produce the mixture of oligosaccharides, wherein the mixture of oligosaccharides has the desired polymolecularity index. The mixture of oligosaccharides of the present invention can have a polymolecularity index of at least 6 (e.g., at least 8, at least 10, or at least 15). Polymolecularity index is described in AU 1999/63030 A1.

The present invention further provides a process for preparing a mixture of oligosaccharides, comprising providing a reaction mixture comprising a saccharide having a degree of polymerization ranging from 1 to 4, and a starch having a degree of polymerization of at least 200; selecting a desired number average molecular weight Mₙ for the mixture of oligosaccharides; selecting extrusion conditions, which, when applied, produce the average molecular weight Mₙ; and extruding the reaction mixture under the extrusion conditions, wherein the extruding imparts sufficient energy and work to derivatize the starch with the saccharide to produce the mixture of oligosaccharides, wherein the mixture of oligosaccharides has the desired average molecular weight Mₙ. Preferably, the number average molecular weight Mₙ is at least about 5000 g/mole (e.g., from about 5000 g/mole to about 10,000 g/mole).

Any suitable starch may be used in conjunction with the invention. Exemplary starches include corn, potato, waxy maize, tapioca, rice, and the like. Chemically, starches are homopolysaccharides that are composed of repeating glucose units in varying proportions. Starch molecules have one of two molecular structures: a linear structure, known as amylose; and a branched structure, known as amylopectin. Amylose and amylopectin associate through hydrogen bonding and arrange themselves radially in layers to form granules. This ratio of amylase to amylopectin varies not only among the different types of starch, but among the many plant varieties within a type. For instance, waxy starches are those that have no more than 10% amylopectin, whereas high amylose starches are composed of essentially 100% amylose. In connection with the present invention, the starch may be a waxy starch, or may be a high amylose starch, or may be any other starch found suitable for use in connection with the invention. A preferred starting material is dent corn starch. One suitable starch is sold under the trademark B200 by Grain Processing Corporation of Muscatine, Iowa. Another is B700 Unmodified/Dried Corn Starch also available from Grain Processing Corporation. The reaction mixture may include other starting materials, such as other starches, oligosaccharides, or other materials.

In some embodiments, the invention contemplates the dextrinization and derivatization of an oligosaccharide in a mixture with the starch. When used, the oligosaccharide preferably is a malto-oligosaccharide. By “malto-oligosaccharide” is contemplated any species comprising two or more saccharide units linked predominantly via 1-4 linkages, and including maltodextrins and syrup solids. Maltodextrins have a dextrose equivalent value (DE) of less than 20, whereas syrup solids have a DE of 20 or greater. In preferred embodiments, at least 50% of the saccharide units in the malto-oligosaccharide are linked via 1-4 linkages. More preferably, at least about 60% of the saccharide units are linked via 1-4 linkages; and even more preferably, at least about 80% of the saccharide units are so linked. Malto-oligosaccharides may include saccharide species having an odd or even DP value, and may include some dextrose (DP 1). The invention is applicable to derivatization of malto-oligosaccharide species in which at least a portion of the malto-oligosaccharides in the mixture have a DP value greater than 5. Preferably, at least one of the malto-oligosaccharides species in the mixture has a DP value of 8 or more. Most preferably, at least one species has a DP value of at least 10. In preferred embodiments in the invention, at least 70% of the malto-oligosaccharide species in the mixtures have a degree of polymerization greater than 5; even more preferably, at least about 80% of the malto-oligosaccharides species in the mixture have a degree of polymerization greater than 5.

Suitable malto-oligosaccharides are sold as maltodextrins under the trademark MALTRIN® by Grain Processing Corporation of Muscatine, Iowa. The MALTRIN® malto-oligosaccharides are malto-oligosaccharide products, each product having a known typical DP profile. Suitable MALTRIN® maltodextrins may serve as starting materials in accordance with the present invention and include MALTRIN® M040, MALTRIN® M050, MALTRIN® M100, MALTRIN® M150, and MALTRIN® M180. Typical DP profiles of the subject MALTRIN® maltodextrins are set forth in the following table:

Typical DP profile (% dry solids basis)

| DP profile | M180 | M150 | M100 | M050 | M040 |
|---|---|---|---|---|---|
| DP > 8 | 46.6 ± 4% | 54.7 ± 4% | 67.8 ± 4% | 90.6 ± 4% | 88.5 ± 4% |
| DP 8 | 3.9 ± 2% | 4.8 ± 1.5% | 4.5 ± 1.5% | 1.5 ± 1% | 2.0 ± 1% |
| DP 7 | 9.5 ± 2% | 9.1 ± 1.5% | 7.0 ± 1.5% | 1.5 ± 1% | 2.4 ± 1% |
| DP 6 | 11.4 ± 2% | 8.4 ± 1.5% | 6.1 ± 1.5% | 1.4 ± 1% | 1.8 ± 1% |
| DP 5 | 5.9 ± 2% | 4.7 ± 1.5% | 3.3 ± 1.5% | 1.3 ± 1% | 1.3 ± 1% |
| DP 4 | 6.4 ± 2% | 5.5 ± 1.5% | 3.7 ± 1.5% | 1.1 ± 1% | 1.4 ± 1% |
| DP 3 | 8.3 ± 2% | 6.7 ± 1.5% | 4.2 ± 1.5% | 1.0 ± 1% | 1.4 ± 1% |
| DP 2 | 6.2 ± 2% | 4.8 ± 1% | 2.5 ± 1% | 0.8* ± 1% | 0.9* ± 1% |
| DP 1 | 1.8 ± 1.5% | 1.3 ± 1% | 0.7* ± 1% | 0.8* ± 1% | 0.3* ± 1% |

*Minimum Value = 0%

Each of these maltodextrins has at least 45% DP 10 or greater malto-oligosaccharide. Other suitable malto-oligosaccharide starting materials can include other malto-oligosaccharides, such as MALTRIN® M440, MALTRIN® M4510, MALTRIN® M580, MALTRIN® M550, and MALTRIN® M700, as well as corn syrup solids, such as MALTRIN® M200, MALTRIN® M250, and MALTRIN® M360. The malto-oligosaccharides can be ion-exchanged or hydrogenated. One method for hydrogenating mixtures of malto-oligosaccharides is disclosed in published PCT Application WO 99/36442 (to Grain Processing Corporation). The malto-oligosaccharide starting materials further may be derivatized, as disclosed, for instance, in U.S. Pat. No. 6,380,379. The invention is not limited to use in conjunction with the foregoing malto-oligosaccharide species, and indeed, any suitable malto-oligosaccharide may be employed with the starch as a starting material in conjunction with the present invention.

In accordance with another embodiment of the invention, the starting material can include a limit dextrin. Limit dextrins are discussed in more detail in copending application Ser. No. 09/796,027. Alternatively, or in addition thereto, the starting material may be another dextrin that comprises a starch that has been partially hydrolyzed by an alpha amylase enzyme but not to the theoretical or actual limit. Such dextrins are referred to herein as “prelimit dextrins.”

In accordance with some embodiments of the invention, at least a portion of the starting material is hydrogenated. For instance, the invention can comprise a starch starting material and a hydrogenated oligosaccharide. It is suitable, for example, for the invention to comprise a mixture of malto-oligosaccharide species that is catalytically reduced. It has been found, as described in WO 99/36442A1, that when a starting malto-oligosaccharide mixture is catalytically hydrogenated in accordance with the invention, the reduced malto-oligosaccharide mixture thus formed will have a DP profile that is not substantially altered as compared with the DP profile of the starting malto-oligosaccharide mixture. Moreover, it has been found that the resistance to color formation of the reduced malto-oligosaccharide, as measured by the light absorbance thereof, is improved relative to the starting mixture of unreduced malto-oligosaccharides. A liquid mixture of the reduced malto-oligosaccharides will be stable, and, it is believed, relatively more stable than a liquid mixture of unreduced malto-oligosaccharides.

Generally, and as described in more detail in the aforementioned WO 99/36442A1, the hydrogenation of the malto-oligosaccharide may be accomplished in any suitable manner. For example, in one embodiment of the invention, the hydrogenation is accomplished chemically, using sodium borohydride or another hydride donor. Preferably, however, the hydrogenation is accomplished catalytically, in the presence of a metal catalyst suitable for catalyzing the hydrogenation of the polysaccharide in the presence of hydrogen. Examples of suitable hydrogenation catalysts include palladium, platinum, ruthenium, rhodium, and nickel. The metal catalyst may be in the form of the neutral metal, or may be in the form of suitable metal is alloy, oxide, salt, or organometallic species. Preferably, the catalyst is nickel or an activated nickel species, (such as a molybdenum promoted nickel species).

Examples of suitable commercially available catalysts include A-7063 (Activated Metals and Chemicals, Inc.); H07 (Engelhard) Raney™ 3110, 3111, and 3201 (Davison Chemical); and BK113W (Degussa), with the most preferred catalyst being Raney 3110. The catalyst may be employed in any amount effective to catalyze hydrogenation of the polysaccharide species, and preferably is present in an amount ranging from about 0.5 to about 100 or even from about 0.5 to about 10 (W/w polysaccharide) in the reaction mixture.

The hydrogenation of the malto-oligosaccharide or other polysaccharide is accomplished under pressures and temperatures suitable to maintain the DP profile thereof. The reaction pressure preferably ranges up to about 1500 psi. More preferably, the pressure ranges from about 200 psi to about 1200 psi; even more preferably the pressure ranges from about 400 psi to about 700 psi. The reaction temperature preferably ranges from about 50 to about 150° C.; more preferably, the temperature ranges from about 100° C. to about 130° C.; even more preferably, the temperature ranges from about 110° C. to about 120° C.

Hydrogen optionally may be introduced into the reaction vessel at any rate effective to reduce the polysaccharide. Preferably, the vessel is filled with hydrogen, and additional hydrogen is added a purge rate of up to about 2.5 L/min for a 2.0 L reaction vessel.

The hydrogenation reaction may take place in any medium suitable to effectuate the hydrogenation of the saccharide mixture. Preferably, the reaction takes place in an aqueous medium, under pH conditions suitable for the hydrogenation reaction to proceed. The pH of the medium preferably ranges from about 3.5 to about 8.5, more preferably from about 4.5 to about 6.5, and even more preferably from about 5 to about 6. The invention is generally contemplated in some embodiments to comprise the step of catalytically reducing a saccharide mixture in aqueous solution at the specified pH ranges. For example, the invention encompasses a method comprising the steps of providing an oligosaccharide or oligosaccharide mixture, such as a malto-oligosaccharide mixture, and catalytically hydrogenating the mixture in aqueous solution at a pH ranging from about 3.5 to about 8. To ensure adequate hydrogenation under these temperatures and pressures, the reaction mixture should be vigorously agitated. Hydrogenation should proceed for a time sufficient for the DE value of the polysaccharide mixture to be reduced to essentially zero. In preferred embodiments of the invention, the reaction time ranges from about 0.5 hours to about 72 hours, more preferably, from about 1 hour to about 8 hours, even more preferably, about 2 to about 4 hours.

The reaction may be performed in a catalytic bed containing the metal catalyst. In accordance with this embodiment of the invention, the saccharide and hydrogen are continuously introduced into the reaction bed under conditions sufficient to reduce the DE of the saccharide to a value of essentially zero while maintaining the DP profile. The temperature and pressure conditions in the catalytic bed may be substantially as hereinbefore described.

It has also been found that reduced malto-oligosaccharides prepared as described herein have low light absorbance values. For example, in preferred embodiments of the invention, the absorbance of the reduced malto-oligosaccharide is less than about 0.25; more preferably, the absorbance is less than about 0.15, after holding a solution of the malto-oligosaccharide at 750 C and pH 10 for two hours. As used herein, the absorbance refers to the absorbance at 450 mn of a 10% solution of the malto-oligosaccharide, as measured in a 1 cm cell. In contrast, the UV absorbance 30 of MALTRIN® M100, a product which has a DE of about 10, is about 0.73 after being treated under the same conditions. The surprisingly low light absorbance of the reduced malto-oligosaccharides of the present invention after stressing under the aforementioned reaction conditions indicates an enhanced resistance to color formation.

When used, the starch and other higher order saccharide may be present in any suitable ratio to one another. For instance, the starch (or mixture of starches) may be present in an amount of 100% by total weight of starch in combination with saccharide having a DP greater than four, or another amount, such as 90%, 80%, 70%, 60%, 50%, 40%, 30%, 20%, or 10%. It is presently believed preferred to employ 100% starch (which includes a mixture of starches) relative to the amount of other higher order carbohydrate in the starting material. The starches in a mixture of starches may be employed in any suitable ratio to one another.

In accordance with the invention, the starch, or mixture of starches, or mixture of starch with other saccharides, as described hereinabove is dextrinized in the presence of a lower molecular weight saccharide, i.e., a saccharide having a degree of polymerization ranging from 1 to 4. Mixtures of malto-oligosaccharides typically include some DP 1-4 saccharides, and, when used in combination with a starch, such malto-oligosaccharides provide some such saccharide for use in the derivatization of the starch. In most cases additional saccharide should be added.

Preferably, the saccharide is dextrose, optionally in combination with one or more other saccharides, such as maltose, maltotriose or maltotetraose. The dextrose may be in the form of a monohydrate. If a mixture of saccharides is employed, the average DP of the mixture should be in the range of 1 to 4, preferably 1 to 3, and even more preferably 1 to 2. Mixtures of saccharides that can be employed include MALTRIN® M250 and MALTRIN® M360. It is contemplated that these latter products, which include some lower molecular weight saccharides and some oligosaccharides having a DP greater than four, may themselves be extruded as part of the starting material and thus may be deemed themselves to be a mixture of the saccharide and oligosaccharides. Alternatively, the derivatizing saccharide may be maltose, maltotriose or maltotetraose in the presence or absence of dextrose. However, dextrose is the preferred saccharide. Most preferably, the dextrose is present as 100% of the weight of the saccharides having a DP ranging from 1 to 4, but dextrose may be present in any relative amount, such as 90%, 80%, 70%, 60%, 50%, 40%, 30%, 20% or 10% by weight of saccharaide having a DP from 1 to 4. Preferably, if a mixture of saccharides having a DP of 1 to 4 is employed, the saccharide includes dextrose or maltose in an amount of at least 50% by weight of the total saccharides having a DP of four or less. It has been found that in the derivatization reaction, the dextrose serves as a processing aid in addition to being a reactant. In some embodiments of the inventions, a hydrogenated starch hydrolyzate, preferably sorbitol, but also possibly maltitol or a higher order hydrogenated starch hydrolyzate, is used in connection with the low-order saccharide. Such a hydrogenated starch hydrolyzate serves as a chain terminator to limit the formation of high molecular weight molecules and also serves as a plasticizer and processing aid in connection with the reaction. When a hydrogenated starch hydrolyzate is used, it preferably is present in an amount ranging from about 50 to about 95% by weight of the added saccharide component.

The reaction preferably is catalyzed using an acid, which is present in an amount ranging from about 0.01 to about 1.5% by weight, preferably about 0.1 to about 0.5% by weight of the total reaction mixture. The preferred acid is citric acid, which should be used in an amount ranging of about 0.125% by weight of the total reaction mixture. Other suitable acids include acetic acid, adipic acid, fumaric acid, gluconic acid, lactic acid, malic acid, phosphoric acid, and tartaric acid.

The higher molecular weight saccharide, (i.e. the starch, or mixture of starch with other material such as malto-oligosaccharides) and lower molecular weight saccharide preferably are present in a ratio of about 4:1 (high molecular weight:low molecular weight). It is contemplated that the 4:1 ratio is approximate, and may be varied depending on the reactants chosen and/or the reaction conditions employed. It has been observed that as the molecular weight of the oligosaccharide increases, the amount of dextrose or other lower molecular weight saccharide also should increase. More generally, the amount of dextrose or the lower molecular weight saccharide should be at least about 10% to about 30% by total saccharide weight. “Total saccharide weight” is deemed to include the combined weight of all of the low molecular weight saccharides, the oligosaccharides, and the polysaccharides in the reaction mixture, and includes hydrogenated, derivatized, or otherwise modified materials such as those discussed hereinabove. There is believed to be no operatively limiting upper range of the dextrose or other lower molecular weight saccharide, and thus this material may be present in an amount, for instance, up to 99% by total saccharide weight. It is believed, however, that when the dextrose or other lower molecular weight saccharide is present in an amount greater than about 50% by total saccharide weight, the quality of the color of the extruded mixture may be degraded, and the molecular weight may become lower than desired. In one embodiment, the invention encompasses selecting a desired Mₙ for the oligosaccharide product, the Mn preferably being at least 5,000 g/mol and more preferably being at least 10,000 g/mol, and selecting relative amounts of low molecular weight saccharide and higher molecular weight material (such as starch) and extrusion conditions that will yield a product having the desired Mₙ.

When mixtures of starches are employed, the mixture of starches may be present in amounts of 20-90% by total saccharide weight; in some embodiments, about 77% to about 87%. When mixture of a first starch with an oligosaccharide (such as a malto-oligosaccharide, limit dextrin, hydrogenated maltodextrin, or the like), and/or second starch is employed as a starting material, the amount of such material having a DP of 5 or greater should be present in an amount of about 20%-90%, with the balance comprising the saccharide having a DP of 1-4 (exclusive of acid and any other material in the reaction mixture). In highly preferred embodiments, the low molecular weight saccharide is dextrose, and the high molecular weight materials is starch, and the dextrose is present in an amount ranging from 30% to 50% by total saccharide weight. The most highly preferred reaction mixture includes 50% starch and 50% dextrose by total saccharide weight.

All of the foregoing weight percentages are expressed on a dry solids basis. It has been found that moisture may be present in the reaction mixture without detracting from the derivatization reaction. It is contemplated that moisture may be present in an amount of up to about 50% by weight. Preferably, any moisture is present in a substantially lower amount, such as about 5 to 10% by weight, to permit moisture to be added during extrusion of the mixture of starting materials. In any event, the moisture content of the starting materials is not critical.

In carrying out the invention, the starting materials, which include the starch and any other higher molecular weight saccharide, the saccharide, any hydrogenated starch hydrolyzate, any catalyzing acid, and any other material may be reacted in any suitable fashion to dextrinize the starch or other starting material. The dextrinization should be sufficient to convert at least a portion of the highly digestible 1-4 bonds present in the starting material to other bonds. Generally, the application of heat and/or material energy is necessary to dextrinize the starting material. Most preferably, the starting materials are combined and reacted in an extruder. The extruder can include any conveying device in which temperature, vacuum, water, and the starting materials can be introduced with adequate mixing to result in derivatization. For example, a Wenger TX-57 Twin Screw Extruder can be used to generate an acceptable product. The extruder may be operated under any suitable conditions. Generally, extrusion conditions require barrel temperatures that range from about 25° C. to about 220° C., with the maximum barrel temperature more preferably in a range of about 140° C. to 180° C. The internal sample temperature at the dye head of the extruder can be in a range of 160° C. to 275° C., but preferably remains between the range 190° C. to 230° C. The revolutions permitted for the extruder can vary between 25 and 500 rpm, with optimal conditions in the 300 to 425 rpm range. Vacuum optionally can be applied to the system; if applied, up to 18 inches of mercury (0.4 atm) can be used. The foregoing set of conditions is by no means meant to be exhaustive or limiting, but to the contrary these conditions are provided for general guidance. The actual extruder conditions can vary widely depending on the starting materials and the type of extruder being used. The dextrinized oligosaccharides preferably are formed from the foregoing ingredients in the absence of other ingredients. It is contemplated that other derivatizing agents, other catalysts, or the like could be employed.

The amount of lower molecular weight saccharide should be selected relative to the amount of higher order starting material such that the product that is extruded from the extruder barrel appears as a straw-colored, low-density solid that crumbles and dissolves easily. Preferably, the amount of saccharide chosen is sufficient to yield such product without charring, but insufficient to result in a product that is in liquid form. Excess dextrose will result in poor processing conditions. The exact amount of dextrose chosen in a given extrusion reaction is a matter well within the purview of one of ordinary skill in the art. When a mixture of starch is reacted with the saccharide, the starch is “derivatized,” by which is contemplated the derivatization of at least a portion of higher order materials.

The derivatized oligosaccharide product prepared by the foregoing process is easily solubilized and, in preferred embodiments, requires little downstream processing to substantially reduce the levels of undesired color and flavor components. For example, the product can be dissolved in water and treated with 0.5 to 10% carbon, such as SA-30 carbon from Westvaco, for up to 4 hours at 75° C. The material then may be filtered and otherwise treated, for instance, by spray-drying. Spray-drying of the decolorized material yields an off-white final product with a bland, malto-oligosaccharide taste. Further processing such as chemical bleaching, ion exchange, membrane filtration, or hydrogenation can also be used to improve the final color of the product. If an ion exchanged or hydrogenated-ion exchanged starting material is used, downstream processing to remove color and flavor components may be facilitated or made altogether not necessary.

The resulting product may have a low caloric value relative to dextrose. It is believed that this is because the product will be unaffected by amylolytic enzymes such as amylo-1-4-glucosidases, amylo-1-4, 1-6-glucosidases, amylo-1-4-dextrosidases, and amylo-1,4 maltosidases, as well as alpha-beta-glucosidases, sucrase, and phosphorylase. Thus, the product may be substantially inert to digestion by mammalian enzymes, although mammalian intestinal flora may be able to ferment a portion of the product and make fermentation products available for digestion. The product alternatively may be substantially digestible, but digestible slowly relative to glucose. It is believed that relatively low levels of chemical modification of the starting material will produce a product having some non 1-4 linking bonds, (e.g., 1-2, 1-3, or 1-6 bonds) that are resistant to enzymatic degradation in the digestive system. The majority of the bonds will be subject to enzymatic hydrolysis. Because of the random nature of the new bonds that are formed, the overall product will be digested slowly relative to the starting material (and relative to glucose) due to less enzymatic recognition of the hydrolysable segments of the material.

Surprisingly, it has been found that derivatization of starches (or starch mixtures) with a saccharide having a degree in polymerization of 1-4 provides a product that has low color even before being subjected to decolorization using activated carbon, or other means. It has been found that a oligosaccharide product having a molecular weight from 10,000-150,000, more typically, 15,000-130,000 and having a DE from 15 and 40 can be prepared. The Minolta color value can be greater than 85, and can be in the range from 88-95, prior to decolorization using activated carbon or other decolorization step. The solubility can be greater than 50% at 25° C.

The product thus prepared is suitable for use in numerous applications. Typical uses are found in low calorie spreadable foods such as jellies, jams, preserves, marmalades, sugar-fruits, compotes, fruit garnish, fillings, and fruit butters; in frozen food compositions, including ice cream, iced milk, sherbet, and water ices; in baked goods, such as cakes, cookies, pastries, and other foodstuff containing wheat or other flour; in icings, candy, and chewing gum; in beverages, such as non-alcoholic soft drinks, root extracts, fruit or vegetable juices, or mineral water; in syrups; in toppings, sauces, and puddings; in salad dressings; and so forth. The invention finds particular use as a bulking agent for dry low calorie sweeteners such as saccharin, sucralose, or aspartame. The product also finds use as a carrier or excipient. More generally, the product may be used as a bulking agent for products such as soaps, cosmetics, food products, animal feeds, and so forth. It is further contemplated that the product may find other uses. For instance, in embodiments of the invention where the product is digestible slowly, the product may be used in sports and nutritional drinks and solid food products such as energy bars. The product may be used in products for individuals with diabetes.

Moreover, the product thus prepared is suitable for use as texturizing agents, thickening and/or gelling agents, emulsifying agents, filling or encapsulating agents, particularly in food products, in pharmaceutical or veterinary products, and in sugar-free confectioneries (e.g., chewy pastes, caramels, toffees, chocolates, fudges and nougats), which may comprise viscosity-promoting agents (gum arabic, gelatin, modified starches, maltodextrins, carrageenans, agar, pectin, and the like), humectants (sorbitol, glycerin), egg white, and flavorings. Moreover, the product can be used in compositions intended to be ingested by humans and animals, e.g., those administered orally, e.g., soups, fiber-enriched fruit-based compositions, fiber-enriched drinks, e.g., fiber-enriched low-calorie drink (e.g., fiber-enriched soft drinks), mayonnaise, biscuits, lozenges, preparations based on milk, fermented milks, and foodstuff fermentations. The fermented food compositions at which the present invention is directed can be of animal or vegetable origin and can also be intended for animal nutrition, particularly as silage-making compositions. The product can be in the form of dessert creams or yogurts directly consumed or which can be administered by a tube. Moreover, the products find use in dietetic or hygiene applications such as elixirs, cough syrups, tablets or pills, hygienic solutions for oral cavity, toothpastes and tooth gels.

Sports drinks, for example, are available in several compositions. In one embodiment, the composition is a ready-to-drink aqueous solution that can be packaged in single serving or larger containers. The components are mixed together in sterile, filtered, or carbonated water and packaged for sale. In another embodiment, the components are mixed in an aqueous solution in a concentrated form. A portion of the concentrated solution is then mixed with a pre-measured amount of water to prepare the beverage. In another embodiment, the composition is a dry powdered form in which the dried components are mixed together and milled or mixed in aqueous solution and dried by one of the methods described below. A portion of the dried components is mixed with a pre-measured amount of water to prepare the beverage. The dry powder may be loose or fashioned into tablets which can be easily added to a pre-measured amount of water to prepare the beverage.

Sports drinks can additionally comprise, other sugars, e.g., trehalose. Other suitable carbohydrates include mono- di- and polysaccharides. Suitable monosaccharides include, but are not limited to, fructose, mannose, glucose arid galactose. Suitable disaccharides include, but are not limited to, sucrose, maltose and lactose. Suitable polysaccharides include, but are not limited to, maltodextrins and those described in European Patent Specification Publication No. 223,540.

Moreover, sports drinks can comprise suitable salts, which include, but are not limited to, sodium, potassium, magnesium and calcium. European Patent Application Publication No. 587,972 provides an extensive discussion of such salts and suitable concentrations thereof. Suitable sources of the salts include, but are not limited to, sodium chloride, potassium phosphate, potassium citrate, magnesium succinate and calcium pantothenate. Salts are optional, and, as discussed herein, are primarily beneficial in increasing fluid intake by the intestinal tract. Thus, the amount of salts added is preferably suitable to affect an increase in fluid intake without resulting in an unpalatable drink.

In addition to carbohydrates and salts, the sports drink may contain various other nutrients. These include, but are not limited to, vitamins, minerals, amino acids, peptides and proteins. Suitable vitamins include, but are not limited to, vitamin C, the B vitamins, pantothenic acid, thiamin, niacin, niacinamide, riboflavin, iron and biotin. Minerals include, but are not limited to, chromium, magnesium and zinc. Preferably, amino acids are included rather than peptides and proteins which require digestion prior to absorption. Suitable amino acids include, but are not limited to, the twenty amino acids utilized by humans. U.S. Pat. No. 4,871,550 discusses preferred amino acids. The effective amounts of the various nutrients are known in the art and are not described in detail herein. Other ingredients include, but are not limited to, coloring, flavor, artificial sweeteners and preservatives may also be added. Suitable amounts and types of all ingredients described herein are known in, the art and are not described in detail herein. It is within the skill of one in the art to prepare a beverage formulation having suitable concentrations of all the components.

Energy bars can additionally comprise nutrients, such as calcium, vitamin D, vitamins B12, folic acid, B6, niacin, C or E, iron and zinc. Moreover energy bars can comprise lipoic acid and carnitine, optionally in combination with coenzyme Q10 and/or creatine, in a timed release formulation to provide a steady supply of the nutrients to the mitochondria which work 24 hours a day. Such additional components can be in any suitable form, e.g., coating a core comprising the micronutrient(s) and excipients (coated system) and incorporating the micronutrient(s) into a matrix (matrix system). Coated systems involve the preparation of product-loaded cores and coating the cores with release rate-retarding materials. Product-loaded cores can be formulated as microspheres, granules, pellets or core tablets. There are many known core preparation methods, including, but not limited to, 1) producing granules by top spray fluidized bed granulation, or by solution/suspension/powdering layering by Wurster coating; 2) producing spherical granules or pellets by extrusion-spheronization, rotary processing, and melt pelletization; 3) producing core tablets by compression and coating with a release rate-retarding material; 4) producing microspheres by emulsification and spray-drying.

Matrix systems embed the micronutrient in a slowly disintegrating or non-disintegrating matrix. Rate of release is controlled by the erosion of the matrix and/or by the diffusion of the micronutrient(s) through the matrix. In general, the active product substance, excipients and the release rate-retarding materials are mixed and then processed into matrix pellets or tablets. Matrix pellets can be formed by granulation, spheronization using cellulosic materials, or by melt pelletization using release retardant materials, while matrix tablets are prepared by compression in a tablet press. An example of a cellulosic material is hydroxypropylmethylcellulose as the release rate-retarding material.

Coated or matrix pellets can be filled into capsules or compression tabletted. The rate of release can be further modified by blending coated or matrix pellets with different release rates of the same product to obtain the desired product release profile. Pellets containing any of lipoic acid, carnitine, coenzyme Q10 or creatine can be blended to form a combination product.

More generally, the invention is also contemplated to be suitable for use in connection with the uses disclosed in published U.S. Patent Application Nos. 2003/0077368 (entitled “Fibre-enriched drinks”); 2003/0039740 (“Composition for enteral nutrition comprising fibres”); 2002/0192355 (“Fibre-enriched table sweeteners”); 2002/0192344 (“Process for preparing a low-calorie food”); 2002/0182299 (“Process for manufacturing fibre-enriched fruit-based compositions and compositions thus obtained”); and 2002/0136798 (“Carbon containing additive for foodstuff fermentations and food compositions containing it”) and in published Australian Application No. AU 1999/63030 A1 (“Branched maltodextrins and method of preparing them”). The materials disclosed in connection with the present application may be substituted for the materials purportedly described in the foregoing publications.

The following examples are provided to illustrate the present invention, but should not be construed as limiting the scope of the invention. Examples 1-35 do not illustrate dextrinization of a starch, but are provided for convenient reference.

### EXAMPLES 1-18

Preparation of Dextrinized Saccharide-Derivatized Oligosaccharides. These examples illustrate the preparation of various saccharide-derivatized oligosaccharides. A blend of maltodextrins/anhydrodextrose/citric acid (87.5%/12.5%/1.0%) was made by mixing 1312.5 grams of MALTRIN® M100 and other MALTRIN® products with 187.5 grams of anhydrodextrose and 15 grams of citric acid. These materials were thoroughly mixed in a Hobart mixer. The resulting blend was then manually fed into an 18 mm twin screw Leistritz extruder. The extruder barrel temperature was monitored in six zones, according to the following table:

| | |
|---|---|
| Zone 1 | 32° C. |
| Zone 2 | 81° C. |
| Zone 3 | 180° C. |
| Zone 4 | 201° C. |
| Zone 5 | 201° C. |
| Zone 6 (die head) | 198° C. |

Low shear extruder screws were used. The extruder screw speed rate was 100 rpm. A single, 3 mm dye opening was used at the die head. The percent motor load for the extruded sample was 55%.

In each instance, a straw-colored solid material was extruded. The material was allowed to cool and ground to a golden yellow powder. Each sample was analyzed for molecular weight, percentage digestibility, and color. Molecular weight calculations were done via HPLC-SEC TRISEC (VISCOTEK® Corporation, Houston Tex.). For control purposes, dextrose was extruded. The products were prepared according to the following table:

Percent Composition

| | | | Dextrose | Citric | | Shaft | |
|---|---|---|---|---|---|---|---|
| | | MD wt. % | wt. % | Acid | Max. | Speed | % Motor |
| Example | MD Type | dsb | dsb | wt. % | Temp. ° C. | RPM | Load |
| Control | N/A | 0 | 100 | 1 | 200 | 25 | 39 |
| Control 2 | N/A | 0 | 100 | 1 | 220 | 25 | 16 |
| 1 | M360 | 50 | 50 | 1 | 200 | 100 | 38 |
| 2 | M250 | 50 | 50 | 1 | 200 | 100 | 38 |
| 3 | M250 | 93.75 | 6.25 | 1 | 200 | 100 | 40 |
| 4 | M250 | 96.875 | 3.125 | 1 | 200 | 100 | 55 |
| 5 | M250 | 100 | 0 | 1 | 200 | 200 | 55 |
| 6 | M200 | 100 | 0 | 1 | 200 | 200 | 75 |
| 7 | M200 | 50 | 50 | 1 | 200 | 100 | 30 |
| 8 | M180 | 50 | 50 | 1 | 200 | 100 | 40 |
| 9 | M150 | 50 | 50 | 1 | 200 | 100 | 55 |
| 10 | M100 | 50 | 50 | 1 | 200 | 100 | 55 |
| 11 | M100 | 75 | 25 | 1 | 200 | 100 | 38 |
| 12 | M100 | 87.5 | 12.5. | 1 | 200 | 100 | 55 |
| 13 | M100 | 93.75 | 6.25 | 1 | 180 | 100 | 75 |
| 14 | M100 | 96.875 | 3.125 | 1 | 200 | 100 | 55-60 |
| 15 | M070 | 50 | 50 | 1 | 200 | 100 | 45 |
| 16 | H-M180 | 50 | 50 | 1 | 180 | 100 | 40 |
| 17 | M040 | 50 | 50 | 1 | 200 | 100 | 52 |
| 18 | M040 | 87.5 | 12.5 | 1 | 200 | 100 | 50 |

MD = maltodextrin type
DE = dextrose equivalent
H-M180 - hydrogenated M180

Percentage maltodextrin and dextrose were expressed on a dry solids basis per total weight (maltodextrin and dextrose). The dextrose value represents dextrose added to the malto-oligosaccharide.

Upon analysis, the following results were obtained:

| | | Molecular eight | | | |
|---|---|---|---|---|---|
| Example | DE | Mw | Mn | % digest | UV 420 Color |
| Control | 15.4 | 1860 | 1050 | 7.54 | 2.6 |
| Control 2 | 6.8 | 3700 | 790 | 4.58 | 14.2 |
| 1 | 21.7 | 3010 | 900 | 24.77 | 9.1 |
| 2 | 17.3 | 2940 | 1390 | 16.75 | 27 |
| 3 | 11.2 | 5470 | 2150 | 9.24 | 58.5 |
| 4 | 10.6 | 5720 | 870 | 9.42 | 63.3 |
| 5 | 13.8 | 3390 | 1350 | 35.3 | 14.2 |
| 6 | 11.9 | 6500 | 950 | 14.28 | 118 |
| 7 | 14.6 | 4530 | 740 | 18.71 | 19.5 |
| 8 | 18 | 4980 | 1300 | 21.67 | 10.3 |
| 9 | −18.9 | 4510 | 1890 | 22.11 | 6.8 |
| 10 | 13.2 | 5050 | 630 | 14.42 | 13.8 |
| 11 | 14 | 4850 | 1770 | 22.44 | 9.7 |
| 12 | 11.8 | 4700 | 2150 | 16.59 | 14.8 |
| 13 | 10.2 | 5600 | 2250 | 7.23 | 52.8 |
| 14 | 10.6 | 7650 | 2350 | 5.12 | 136 |
| 15 | 12.8 | 5280 | 1610 | 13.83 | 14.6 |
| 16 | 21.8 | 4780 | 460 | 49.42 | 2.03 |
| 17 | 12.9 | 5290 | 1360 | 13.61 | 17.2 |
| 18 | 10.5 | 6440 | 2450 | 6.82 | 96 |

% digest = 3 hour digestibility adapted from J. S. white et al., J. Food Sci., Vol. 53, No. 4, 1988, pp. 1204-1207
UV 420 color = UV 420/% solids

As seen, a wide variety of combinations of dextrose, citric acid and malto-oligosaccharide can be used to produce low-calorie oligosaccharides. The foregoing data also demonstrates how dextrose aids in extrusion, inasmuch as samples with little or no added dextrose are very dark and difficult to extrude. (It should be noted that each of the MALTRIN® products contains some dextrose). Samples with high levels of dextrose became hard glasses upon drying, thus making downstream processing more difficult. The best results were seen when MALTRIN® M100 and 25% or 12.5% added dextrose was used.

All of the samples incorporating the product of the invention had a higher average molecular weight and number average molecular weight than samples that were extruded only with glucose and citric acid.

### EXAMPLES 19-24

This example illustrates the effect of varying the level of citric acid catalyst in the preparation of dextrinized oligosaccharides.

A mixture of MALTRIN® M100/dextrose monohydrate/citric acid (the dry solid weight ratio of maltodextrin: dextrose being 4:1) was made by mixing 640 lbs of MALTRIN® M100 with 160 lbs of dextrose monohydrate and citric acid. The resulting blend was then automatically fed into a 57 mm twin screw Wenger TX-57 extruder at a rate of 111 lbs per hour. Water was also fed to the extruder barrel at a rate of 12 lbs per hour. The total moisture level of the feed was 18% (7% for the starting material, 11% from added to the extruder water). The extruder barrel temperature was monitored in five zones, according to the following table:

| | |
|---|---|
| Zone 1 | 57° C. |
| Zone 2 | 62° C. |
| Zone 3 | 59° C. |
| Zone 4 | 172° C. |
| Zone 5 (die head) | 172° C. |

The internal sample temperature at the die head was approximately 200 to 210° C. Low shear extruder screws were used. The extruder screw speed rate was 401 rpm. A single, 17 mm dye opening was used at the die head. The percent motor load for the extruded sample was 56%. A vacuum of 13 inches of water (0.57 atm) was used. The following table represents the ingredients and conditions employed.

In each case, the extruded product was a puffy, golden yellow solid material. The material was allowed to cool and ground to a golden yellow powder. The samples were analyzed yielding the following results. Color measurements are dyed on the international standard promulgated by the Commission Internationale d'Eclairage (CIE)

| | | | | | Max | Shaft | | | | |
|---|---|---|---|---|---|---|---|---|---|---|
| | MD | | | Citric | Temp. | Speed | % Motor | | | |
| Example | type | MD % | Dextrose | Acid | ° C. | RPM | Load | DE | % Digest | Color L |
| 19 | M100 | 80 | 20 | 0 | 199 | 401 | 56 | 14.9 | 56.5 | 88 |
| 20 | M100 | 80 | 20 | 0.075 | 209 | 401 | 60 | 6.1 | 24.5 | 80 |
| 21 | M100 | 80 | 20 | 0.125 | >179 | 401 | 56 | 6 | 23.5 | 79 |
| 22 | M100 | 80 | 20 | 0.25 | >182 | 401 | 58 | 5.8 | 21.6 | 79 |
| 23 | MIN | 80 | 20 | 0.5 | 204 | 145 | 48 | 6.1 | 21.1 | 75 |
| 24 | M100 | 80 | 20 | 1 | 208 | 144 | 58 | 6.7 | 22.9 | 76 |

As seen, low levels of citric acid can be used to obtain the desired levels of digestibility. Citric acid aids in reducing digestibility and color formation.

### EXAMPLE 25

A sample of saccharide-derivatized oligosaccharides was prepared in accordance with Example 22. Five hundred grams of the product were slurried in warm water so that the total solids content was approximately 25%. Carbon SA-30 (Westvaco, Covington, Va.), 25 grams (5%) was added and the mixture was heated to 75° C. and held at this temperature for 4 hours. The solution was filtered through a celite bed to yield a yellow solution (Gardner Color=3, original color=9). The solution was then spray-dried on a Yamato lab spray drier to give 365 grams of an off-white product. The off-white powder had a Minolta L color value of 95 (compared with an initial value of 79). Chemical analysis of the product is shown in the table below:

| | Before | After |
|---|---|---|
| | carbon treatment | carbon treatment |
| 3 hr % Digest # | 18.5 | 18.2 |
| 24 hr % Digest # | 21.6 | 20.6 |
| Dextrose | 1.50 | 1.46 |
| 2DE | 5.81 | 5.42 |
| Citric Acid | 0.209 | 0.20 |
| Levoglucosan | 1.57 | 1.49 |
| 5-HMF* | 0.312 | 0.11 |
| Ash | 0.17 | 0.39 |
| Color: | | |
| L | 79.4 | 95.1 |
| a | −1.4 | −6.1 |
| b | 25.5 | 12.0 |
| VISCOTEK | | |
| Mn | 990 | 1,730 |
| Mw | 9,890 | 7,920 |

# Adapted from J. S. White et al.
*5-HMF = 5 hydroxymethyl furfural

As seen, carbon treatment removes color and 5-HMF, but otherwise does not essentially change the material. The flavor of the product is also greatly improved, as undesired off-flavors imparted by 5-HMF are essentially completely removed by the carbon treatment. The level of leveoglucosan, which can impart bitterness, also was reduced.

The decolored product, two polydextrose products, and a FIBERSOL product were obtained and evaluated. The results are shown on the following table:

| | | LITESSE III | | Reduced Calorie |
|---|---|---|---|---|
| | Polydextrose | Polydextrose | FIBERSOL-2 | Oligosaccharide |
| Dextrose Equivalent | 8.4 | 0.18 | 13.4 | 5.4 |
| Free Glucose | 3.70 | 0 | 2.07 | 1.46 |
| Levoglucosan | 1.26 | 1.42 | 0.20 | 1.49 |
| 5-HMF | 0.68 | 0.35 | N.D.** | 0.11 |
| Citric Acid | 0.66 | 0.002 | 0 | 0.1 |
| Color L Value | 93.80 | 96.36 | 94.71 | 95.05 |
| (White) | | | | |
| Color b | 13.68 | 6.11 | 12.38 | 12.00 |
| Value(Yellow) | | | | |
| Molecular | 530 | 190 | 660 | 1,730 |
| Weight(Mn) | | | | |
| Molecular | 1,300 | 1,050 | 2,620 | 7,920 |
| Weight(Mw) | | | | |
| Highest Detectable | 4 | 4 | 9 | 11 |
| Oligosaccharide* | | | | |
| 24 Hour Digestibility | 5.7 | 6.2 | 7.4 | 21.0 |
| (%) | | | | |

*As detected by capillary electrophoresis
**Not determined

It is thus seen that the product of the invention is higher in molecular weight and comparable in color to polydextrose and FIBERSOL commercial products. Because of this relatively increased molecular weight, the product of the invention more closely resembles a maltodextrin. The product thus suitable for use in a wider range of applications.

### EXAMPLE 26

A sweetener is prepared by blending 965 grams of the spray-dried product of Example 25 with 35 grams calcium saccharin.

### EXAMPLE 27

A sweetener is prepared by blending 700 g of the spray-dried product of Example 25 with 300 g of sucralose.

### EXAMPLE 28

A pharmaceutical formulation is prepared by blending 10 grams acetaminophen with 100 grams of the spray-dried, carbon treated product prepared in accordance with Example 25. The resulting mixture is granulated and encapsulated.

### EXAMPLE 29

A 70/30/1 Limit Dextrin/Dextrose (anhydrous)/citric acid blend was made by mixing 700 g of limit dextrin with 300 g of anhydrous dextrose and 10 g of citric acid thoroughly in a Hobart mixer. The resulting blend was then manually fed into an 18 mm twin screw Leistritz extruder. The extruder barrel temperature was monitored in 6 zones according to the following table:

| | |
|---|---|
| Zone 1 | 50° C. |
| Zone 2 | 160° C. |
| Zone 3 | 180° C. |
| Zone 4 | 200° C. |
| Zone 5 | 200° C. |
| Zone 6 (die head) | 200° C. |

Low shear extruder screws were used. The extruder screw speed rate was 200 rpm. A single, 3 mm die opening was used at the die head. The motor load for the extruded sample was 50%. An off-white solid material was extruded. The material was allowed to cool, and ground to a off-white powder. The in vitro digestibility of the sample was 62% after 2.5 hours of enzyme treatment.

### EXAMPLE 30

Example 29 was repeated, except that the extruder screw speed was 100 rpm. The motor load was 75%. A light yellow solid material was extruded, was allowed to cool, and was ground to a light yellow powder. The in vitro digestibility of the sample was 67% after 2.5 hours of enzyme treatment.

### EXAMPLE 31

Example 30 was repeated, except that the motor load was 50%. An off-white solid material was extruded, was allowed to cool, and was ground to an off-white powder. The in vitro digestibility of the sample was 43% after 2.5 hours of enzyme treatment.

### EXAMPLE 32

Example 31 was repeated, except that the extruder screw speed was 200 rpm. The motor load remained at 50%. An off-white solid material was extruded, was allowed to cool, and was ground to an off-white powder. The in vitro digestibility of the sample was 43% after 2.5 hours of enzyme treatment Thus, it is seen that the invention provides a product that is improved in many respects over known products such as polydextrose. The product of the invention finds applicability as a bulking agent and in numerous other uses.

### EXAMPLE 33

A mixture of MALTRIN® M180/hydrogenated MALTRIN® M180/dextrose monohydrate/citric acid (the dry solid weight ratio of maltodextrin:hydrogenated maltodextrin:dextrose being 2:2:1) is made by mixing 320 lbs of MALTRIN® M180, 320 lbs of hydrogenated MALTRIN® M180, 160 lbs of dextrose monohydrate and citric acid. The hydrogenated maltodextrin is prepared according to WO 99/36442. The resulting blend is then automatically fed into a 57 mm twin screw Wenger TX-57 extruder at a rate of 111 lbs per hour. Water is also fed to the extruder barrel at a rate of 12 lbs per hour. The total moisture level of the feed is 18% (7% for the starting material, 11% from added to the extruder water). The extruder barrel temperature is monitored in five zones, according to the following table:

| | |
|---|---|
| Zone 1 | 57° C. |
| Zone 2 | 62° C. |
| Zone 3 | 59° C. |
| Zone 4 | 172° C. |
| Zone 5 (die head) | 172° C. |

The internal sample temperature at the die head is approximately 200 to 210° C. Low shear extruder screws are used. The extruder screw speed rate is 401 rpm. A single, 17 mm dye opening is used at the die head. The percent motor load for the extruded sample is 56%. A vacuum of 13 inches of water (0.57 atm) is used. The extruded solids are allowed to cool and ground to a powder.

### EXAMPLE 34

Example 33 is repeated, except that hydrogenated MALTRIN® M100 is used in place of the hydrogenated MALTRIN® M180. The hydrogenated maltodextrin is prepared according to WO 99/36442. The extruded solids are allowed to cool and ground to a powder.

### EXAMPLE 35

Example 33 is repeated, except that unmodified corn starch (commercially available from Grain Processing Corporation) is used in place of the hydrogenated MALTRIN® M180. The hydrogenated maltodextrin is prepared according to WO 99/36442. The extruded solids are allowed to cool and ground to a powder.

### EXAMPLE 36

Four different combinations of B700 Unmodified Flash-Dried Corn Starch available from Grain Processing Corporation, Muscatine, Iowa, and dextrose were extruded with 1.0% citric acid catalyst, and the four different combinations of B700 and dextrose were also extruded with 0.5% citric acid catalyst. The four different B700/dextrose combinations are summarized in the following table.

| | |
|---|---|
| 50% B700 @ 100% solids | 50% Dextrose @ 100% solids |
| 50% B700 @ 100% solids | 50% Dextrose @ 91.27% solids |
| 50% B700 @ 90.28% solids | 50% Dextrose @ 100% solids |
| 50% B700 @ 90.28% solids | 50% Dextrose @ 91.27% solids |

The extrusions of each of the eight different mixtures (four combinations at two different catalyst levels) were carried out at two different extruder speeds of 50 rpm and 100 rpm to produce a total of sixteen different products. The extrusions were carried out at 200° C. The mixtures were extruded to give flowable molten products at exceptionally low motor loads on an 18 mm Leistritz Twin Screw Extruder equipped with low shear screws.

Reaction mixtures, extrusion conditions, and analyses of the products and starting mixtures are tabulated below.

Co-Extrusion of Unmodified Starch and Dextrose with 1.0% Citric Acid

| | Product | | | | | |
|---|---|---|---|---|---|---|
| | AS | A | A1 | BS | B | B1 |
| Ingredients | | | | | | |
| Bone Dry B700 | 990 g | 990 g | 990 g | 990 g | 990 g | 990 g |
| Commercial B700 | 0 g | 0 g | 0 g | 0 g | 0 g | 0 g |
| Bone Dry Dextrose | 990 g | 990 g | 990 g | 0 g | 0 g | 0 g |
| Dextrose Monohydrate | 0 g | 0 g | 0 g | 1085 g | 1085 g | 1085 g |
| Citric Acid | 20.00 g | 20.00 g | 20.00 g | 20.00 g | 20.00 g | 20.00 g |
| Extrusion Conditions | | | | | | |
| RPM | | 100 | 50 | | 100 | 50 |
| Highest Temperature | | 200° C. | 200° C. | | 200° C. | 200° C. |
| Rate of Production | | 31.3 g/min | 19.7 g/min | | 34.3 g/min | 25.8 g/min |
| Motor Load | | 30% | 32% | | 28% | 30% |
| Back Pressure | | 50 psi | 50 psi | | 50 psi | 50 psi |
| “SME” | | 95.8K | 81.2K | | 81.6K | 58.1K |
| Analysis | | | | | | |
| % Solids | 99.75 | 98.39 | 98.56 | 95.23 | 98.20 | 98.40 |
| % Solubles | 49.59 | 90.71 | 97.17 | 48.20 | 98.52 | 98.71 |
| RVA Final Viscosity @ | 1410 | 1 | 1 | 1522 | 1 | 1 |
| 20% | | | | | | |
| Turbidity @ 0.5% | | 136 | 50 | | 56 | 44 |
| % Dextrose | 49.1 | 9.1 | 8.4 | 48.5 | 18.0 | 14.8 |
| Molecular Wt (M_{w}) | | 30,233 | 24,689 | | 49,889 | 24,858 |
| Carbohydrate Profile | | | | | | |
| DP1 | | 9.9 | 8.3 | | 18.2 | 14.5 |
| DP2 | | 5.6 | 4.9 | | 7.6 | 6.6 |
| DP3 | | | | | | |
| DP4 | | | | | | |
| DP5 | | 4.2 | 4.0 | | 2.5 | 3.0 |
| DP6 | | 3.1 | 3.0 | | 1.4 | 2.0 |
| DP7 | | | | | | |
| DP8 | | 2.6 | 2.6 | | 2.3 | 3.4 |
| DP9 | | 4.1 | 4.2 | | | |
| >DP9 | | 60.6 | 62.7 | | 58.0 | 60.4 |
| Dextrose Equivalent | 50.0 | 19.2 | 17.6 | 55.9 | 27.2 | 26.1 |
| Solution Color | | 0.054 | 0.035 | | 0.086 | 0.080 |
| @.5% pH = 6.8 | | | | | | |
| Minolta L Color | 97.18 | 88.68 | 87.23 | 97.17 | 91.69 | 90.25 |
| Solution pH @ 20% | 2.53 | 2.74 | 2.75 | 2.54 | 2.69 | 2.76 |
| Digestibility after | | | | | | |
| Cooking | | | | | | |
| Digestibility @ 0 hrs. | 43.78 | 8.93 | 8.17 | 36.21 | 17.12 | 14.04 |
| Digestibility @ 0.33 hrs. | 82.41 | 33.02 | 29.72 | 77.25 | 39.54 | 33.91 |
| Digestibility @ 1.0 hrs. | 90.21 | 39.62 | 35.06 | 90.46 | 45.87 | 39.85 |
| Digestibility @ 2.0 hrs. | 90.58 | 40.48 | 36.26 | 91.22 | 47.05 | 41.32 |
| Digestibility @ 4.0 hrs. | 91.36 | 41.53 | 37.97 | 91.40 | 49.07 | 42.69 |

| | Product | | | | | |
|---|---|---|---|---|---|---|
| | CS | C | C1 | DS | D | D1 |
| Ingredients | | | | | | |
| Bone Dry B700 | 0 g | 0 g | 0 g | 0 g | 0 g | 0 g |
| Commercial B700 | 1097 g | 1097 g | 1097 g | 1097 g | 1097 g | 1097 g |
| Bone Dry Dextrose | 990 g | 990 g | 990 g | 0 g | 0 g | 0 g |
| Dextrose Monohydrate | 0 g | 0 g | 0 g | 1085 g | 1085 g | 1085 g |
| Citric Acid | 20.00 g | 20.00 g | 20.00 g | 20.00 g | 20.00 g | 20.00 g |
| Extrusion Conditions | | | | | | |
| RPM | | 100 | 50 | | 100 | 50 |
| Highest Temperature | | 200° C. | 200° C. | | 200° C. | 200° C. |
| Rate of Production | | 41.7 g/min | 33.4 g/min | | 36.0 g/min | 22.1 g/min |
| Motor Load | | 42% | 51% | | 23% | 22% |
| Back Pressure | | 50 psi | 50 psi | | 40 psi | 30 psi |
| “SME” | | 100.7K | 76.3K | | 63.9K | 49.8K |
| Analysis | | | | | | |
| % Solids | 94.89 | 98.06 | 98.29 | 90.62 | 97.86 | 98.23 |
| % Solubles | 48.12 | 96.93 | 96.50 | 46.94 | 93.14 | 98.46 |
| RVA Final Viscosity @ | 2115 | 1 | 1 | 1936 | 1 | 1 |
| 20% | | | | | | |
| Turbidity @ 0.5% | | 75 | 56 | | 80 | 78 |
| % Dextrose | 50.0 | 21.2 | 16.8 | 50.4 | 21.0 | 14.6 |
| Molecular Wt (M_{w}) | | | 21,237 | | 45,405 | 32,228 |
| Carbohydrate Profile | | | | | | |
| DP1 | | 21.9 | 16.7 | | 21.5 | 14.4 |
| DP2 | | 7.8 | 6.9 | | 7.7 | 6.4 |
| DP3 | | | | | | |
| DP4 | | | | | | |
| DP5 | | 2.0 | 2.7 | | 2.0 | 3.0 |
| DP6 | | 1.1 | 1.7 | | 1.1 | 2.0 |
| DP7 | | | | | | |
| DP8 | | 1.9 | 3.2 | | 1.8 | 3.6 |
| DP9 | | | | | | |
| >DP9 | | 55.8 | 59.1 | | 56.6 | 60.7 |
| Dextrose Equivalent | 53.2 | 30.9 | 26.8 | 54.2 | 29.9 | 24.3 |
| Solution Color | | 0.094 | 0.084 | | 0.098 | 0.110 |
| @.5% pH = 6.8 | | | | | | |
| Minolta L Color | 97.53 | 92.11 | 91.22 | 97.21 | 90.55 | 88.31 |
| Solution pH @ 20% | 2.62 | 2.71 | 2.72 | 2.63 | 2.71 | 2.76 |
| Digestibility after | | | | | | |
| Cooking | | | | | | |
| Digestibility @ 0 hrs. | 44.74 | 20.19 | 16.38 | 44.83 | 20.28 | 13.60 |
| Digestibility @ 0.33 hrs. | 79.84 | 44.84 | 37.52 | 81.19 | 44.41 | 33.75 |
| Digestibility @ 1.0 hrs. | 90.49 | 50.41 | 42.50 | 89.05 | 50.42 | 38.86 |
| Digestibility @ 2.0 hrs. | 90.39 | 51.64 | 44.26 | 92.45 | 51.57 | 41.23 |
| Digestibility @ 4.0 hrs. | 93.30 | 54.07 | 46.01 | 92.47 | 53.40 | 42.43 |

S = Starting material

Co-Extrusion of Unmodified Starch and Dextrose with 0.5% Citric Acid

| | Product | | | | | |
|---|---|---|---|---|---|---|
| | ES | E | E1 | FS | F | F1 |
| Ingredients | | | | | | |
| Bone Dry B700 | 990 g | 990 g | 990 g | 990 g | 990 g | 990 g |
| Commercial B700 | 0 g | 0 g | 0 g | 0 g | 0 g | 0 g |
| Bone Dry Dextrose | 990 g | 990 g | 990 g | 0 g | 0 g | 0 g |
| Dextrose Monohydrate | 0 g | 0 g | 0 g | 1085 g | 1085 g | 1085 g |
| Citric Acid | 10.00 g | 10.00 g | 10.00 g | 10.00 g | 10.00 g | 10.00 g |
| Extrusion Conditions | | | | | | |
| RPM | | 100 | 50 | | 100 | 50 |
| Highest Temperature | | 200° C. | 200° C. | | 200° C. | 200° C. |
| Rate of Production | | 32.2 g/min | 30.6 g/min | | 40.7 g/min | 38.5 g/min |
| Motor Load | | 35% | 56% | | 35% | 52% |
| Back Pressure | | 30 psi | 30 psi | | 30 psi | 30 psi |
| “SME” | | 108.7K | 91.5K | | 86.0K | 67.5K |
| Analyses | | | | | | |
| % Solids | 99.69 | 99.39 | 98.47 | 95.24 | 98.23 | 98.32 |
| % Solubles | 48.57 | 92.96 | 92.91 | 50.71 | 97.33 | 92.79 |
| RVA Final Viscosity @ | 1,831 | 1 | 1 | 1,894 | 1 | 1 |
| 20% | | | | | | |
| Turbidity @ 0.5% | | 109 | 49 | | 59 | 56 |
| % Dextrose | 48.08 | 12.87 | 12.46 | 49.35 | 23.09 | 20.49 |
| Molecular Wt (M_{w}) | | 68,007 | 44,697 | | 125,550 | 57,330 |
| Carbohydrate Profile | | | | | | |
| DP1 | | 13.52 | 12.36 | | 23.91 | 21.10 |
| DP2 | | 6.92 | 6.30 | | 7.85 | 7.51 |
| DP3 | | | | | | |
| DP4 | | | | | | |
| DP5 | | 3.80 | 3.52 | | 1.72 | 2.22 |
| DP6 | | 2.52 | 2.37 | | 0.88 | 1.24 |
| DP7 | | | | | 0.50 | 0.62 |
| DP8 | | 1.99 | 1.92 | | 1.60 | 2.04 |
| DP9 | | 3.21 | 3.20 | | | |
| >DP9 | | 57.70 | 59.40 | | 55.15 | 55.76 |
| Dextrose Equivalent | 50.4 | 24.9 | 24.4 | 47.9 | 31.0 | 29.2 |
| Solution Color | | 0.052 | 0.047 | | 0.084 | 0.085 |
| @.5% pH = 6.8 | | | | | | |
| Minolta L Color | 97.15 | 88.31 | 88.01 | 97.32 | 90.74 | 90.29 |
| Solution pH @ 20% | 2.64 | 3.34 | 3.20 | 2.75 | 3.25 | 3.16 |
| Digestibility After | | | | | | |
| Cooking | | | | | | |
| Digestibility @ 0 hrs. | 42.45 | 12.19 | 11.77 | 50.66 | 22.18 | 19.81 |
| Digestibility @ 0.33 hrs. | 81.28 | 37.06 | 34.23 | 81.06 | 48.19 | 44.08 |
| Digestibility @ 1.0 hrs. | 90.21 | 43.13 | 40.63 | 90.84 | 52.96 | 49.42 |
| Digestibility @ 2.0 hrs. | 90.79 | 44.23 | 42.47 | 92.69 | 56.00 | 50.89 |
| Digestibility @ 4.0 hrs. | 92.46 | 45.43 | 43.83 | 92.50 | 58.03 | 53.46 |
| | Product | | | | | |
| | GS | G | G1 | HS | H | H1 |
| Ingredients | | | | | | |
| Bone Dry B700 | 0 g | 0 g | 0 g | 0 g | 0 g | 0 g |
| Commercial B700 | 1097 g | 1097 g | 1097 g | 1097 g | 1097 g | 1097 g |
| Bone Dry Dextrose | 990 g | 990 g | 990 g | 0 g | 0 g | 0 g |
| Dextrose Monohydrate | 0 g | 0 g | 0 g | 1085 g | 1085 g | 1085 g |
| Citric Acid | 10.00 g | 10.00 g | 10.00 g | 10.00 g | 10.00 g | 10.00 g |
| Extrusion Conditions | | | | | | |
| RPM | | 100 | 50 | | 100 | 50 |
| Highest Temperature | | 200° C. | 200° C. | | 200° C. | 200° C. |
| Rate of Production | | 46.8 g/min | 36.5 g/min | | 52.2 g/min | 36.6 g/min |
| Motor Load | | 46% | 53% | | 34% | 47% |
| Back Pressure | | 30 psi | 30 psi | | 30 psi | 30 psi |
| “SME” | | 98.3K | 72.6K | | 65.1K | 64.2K |
| Analyses | | | | | | |
| % Solids | 94.63 | 98.02 | 98.26 | 90.58 | 97.94 | 98.05 |
| % Solubles | 48.37 | 93.64 | 95.65 | 50.00 | 90.83 | 90.01 |
| RVA Final Viscosity @ | 2,654 | 1 | 1 | 2,808 | 1 | 1 |
| 20% | | | | | | |
| Turbidity @ 0.5% | | 97 | 77 | | 114 | 114 |
| % Dextrose | 49.23 | 26.43 | 21.32 | 50.72 | 29.62 | 26.57 |
| Molecular Wt (M_{w}) | | 81,794 | 41,833 | | 26,177 | 32,852 |
| Carbohydrate Profile | | | | | | |
| DP1 | | 26.87 | 21.36 | | 31.77 | 28.25 |
| DP2 | | 7.71 | 7.76 | | 6.87 | 7.24 |
| DP3 | | | | | | |
| DP4 | | | | | | |
| DP5 | | 1.32 | 2.08 | | 0.80 | 1.12 |
| DP6 | | 0.65 | 1.17 | | 0.39 | 0.55 |
| DP7 | | 0.44 | 0.58 | | 0.32 | 0.40 |
| DP8 | | 1.32 | 2.02 | | 0.39 | 1.27 |
| DP9 | | | | | | |
| >DP9 | | 54.17 | 56.42 | | 53.59 | 54.76 |
| Dextrose Equivalent | 47.1 | 34.2 | 30.2 | 49.0 | 39.0 | 36.0 |
| Solution Color | | 0.084 | 0.102 | | 0.120 | 0.126 |
| @.5% pH = 6.8 | | | | | | |
| Minolta L Color | 97.28 | 90.83 | 89.52 | 97.35 | 91.73 | 90.86 |
| Solution pH @ 20% | 2.73 | 3.32 | 3.22 | 2.90 | 2.91 | 3.45 |
| Digestibility After | | | | | | |
| Cooking | | | | | | |
| Digestibility @ 0 hrs. | 44.97 | 25.06 | 19.75 | 44.02 | 28.34 | 25.44 |
| Digestibility @ 0.33 hrs. | 81.70 | 51.55 | 45.21 | 79.19 | 55.37 | 51.15 |
| Digestibility @ 1.0 hrs. | 89.13 | 57.81 | 50.48 | 87.63 | 62.25 | 57.93 |
| Digestibility @ 2.0 hrs. | 90.45 | 59.72 | 52.29 | 87.07 | 63.65 | 59.36 |
| Digestibility @ 4.0 hrs. | 90.27 | 60.87 | 52.72 | 89.77 | 63.67 | 60.82 |

S = Starting material

Minolta L color is measured by a STANDARD ANALYTICAL METHOD, color (Minolta), method no. S-45, available from Grain Processing Corporation, Muscatine, Iowa. Bone dry is 1% moisture or less. Drying is performed in an oven.

All of the extruded products had solubilities greater than 90%. All have very low molecular weights. The digestibilities (after cooking in water) of the starting B700/dextrose mixtures were reduced from about 90% to values less than about 60% in the extrudates. Comparison of the RVA Pasting Curves of the starting material mixtures to those of the extruded products show the extrudates to be totally gelatinized and to be greatly reduced in viscosity. The products are of low color.

### EXAMPLE 37

Low-calorie, water-soluble, low viscosity products were prepared by extruding mixtures containing anhydrous dextrose, bone dry dessicated raw starch, and 1% citric acid. The mixtures were extruded at 200° C. and 100 rpm to give flowable molten products at exceptionally low motor loads on an 18 mm Leistritz Twin Screw Extruder equipped with low shear screws.

The unmodified raw corn starch (B200 Belt-Dried Cornstarch available from Grain Processing Corporation, Muscatine, Iowa)/dextrose extrudate was 96% water soluble, and the acid-modified raw corn starch (B890 Cornstarch available from Grain Processing Corporation, Muscatine, Iowa) B890/dextrose extrudate was 99% water soluble. The digestibility (after cooking in water) of the starting B200/dextrose mixture was reduced from 93% to 35% in the extrudate, and the digestibility (after cooking in water) of the starting B890/dextrose mixture was reduced from 91% to 27% in the extrudate. Comparison of the RVA Pasting Curves of the starting material mixtures to those of the extruded products show the extrudates to be totally gelatinized and to be very greatly reduced in viscosity. The following table contains descriptions of the extrusion experiments, and provides analyses of the products.

| | SUBSTRATE | EXTRUDATE | SUBSTRATE | EXTRUDATE |
|---|---|---|---|---|
| | 1:1 Mixture of | 1:1 Mixture of | 1:1 Mixture of | 1:1 Mixture of |
| | Anhydrous | Anhydrous | Anhydrous | Anhydrous |
| | Dextrose and | Dextrose and | Dextrose and | Dextrose and |
| | Anhydrous B200 + 1% | Anhydrous B200 + 1% | Anhydrous B890 + 1% | Anhydrous B890 + 1% |
| Substrate | Citric Acid | Citric Acid | Citric Acid | Citric Acid |
| Analysis | | | | |
| % Dextrose by YSI* | 55.36% | 8.05% | 55.56% | 4.72% |
| % Digestion @ 3 Hr. | 91.0% | 30.3% | 90.5% | 21.5% |
| % Digestion @ 24 Hr. | 92.6% | 35.4% | 90.6% | 26.9% |
| % Soluble | 51.1% | 96.0% | 50.5% | 99.4% |
| RVA Pasting Curve | FLU 3 @ 20% | FLU 3 @ 20% | FLU 3 @ 50% | FLU 3 @ 50% |
| Peak Viscosity | 619 cP | NO PEAK | 1725 cP | NO PEAK |
| Hot Viscosity @ 95° C. | 77 cP | 17 cP | 537 cP | 26 cP |
| Final Viscosity @ 65° C. | 130 cP | 14 cP | 1014 cP | 47 cP |

*Yellow Springs Instrument Biochemistry Analyzer 2700-S

It is thus seen that an oligosaccharide product may be prepared form the derivatization of starch or other material with a saccharide having a degree of polymerization of 1-4.

While particular embodiments of the invention have: been shown, it will be understood that the invention is not limited thereto since modifications may be made by those skilled in the art, particularly in light of the foregoing teachings. The use of examples and exemplary language should not be read as limiting, and the language used in describing the preferred embodiments likewise should not be construed as limiting. No unclaimed language should be regarded as limiting the scope of the invention. All references cited herein are hereby incorporated by reference in their entireties.

What is claimed is:

1. A mixture of saccharide-derivatized oligosaccharides prepared by extruding a reaction mixture comprising a saccharide having a degree of polymerization ranging from 1 to 4 and a starch, wherein the extruding imparts sufficient energy and work to derivatize the starch with the saccharide.

2. The mixture of claim 1, prepared by extruding a reaction mixture that comprises a mixture of starches.

3. The mixture of claim 1, wherein the starch comprises, corn starch.

4. The mixture of claim 1, wherein the saccharide comprises dextrose.

5. The mixture of claim 4, wherein the dextrose is in the form of a monohydrate.

6. The mixture of claim 1, wherein the saccharide comprises a mixture of dextrose and at least one other saccharide having a degree of polymerization ranging from 1 to 4.

7. The mixture of claim 1, wherein the mixture further comprises a catalyst.

8. The mixture of claim 7, wherein the catalyst is selected from the group consisting of citric acid, acetic acid, adipic acid, fumaric acid, gluconic acid, lactic acid, malic acid, phosphoric acid, and tartaric acid.

9. The mixture of claim 8, wherein the catalyst is citric acid.

10. The mixture of claim 8, wherein the catalyst is present in an amount of about 0.05 to about 5 wt %.

11. The mixture of claim 1, having an Mₙ of at least 5000 g/mol.

12. The mixture of claim 1, having an Mₙ of at least 10000 g/mol.

13. The mixture of claim 1, having a DE ranging from 15 to 40.

14. A process for preparing a mixture of saccharide-derivatized oligosaccharides, comprising:
providing a mixture that comprises a saccharide having a degree of polymerization ranging from 1 to 4, and a starch;
extruding the mixture, wherein the extruding imparts sufficient energy and work to derivatize the starch with the saccharide, to thereby produce a mixture of saccharide-derivatized oligosaccharides.

15. The process of claim 14, comprising extruding the reaction mixture at a speed of 25 to 150 rpm.

16. The process of claim 14, wherein the derivatization is catalyzed with an acid.

17. The process of claim 16, wherein the acid is selected from the group consisting of citric acid, acetic acid, adipic acid, fumaric acid, gluconic acid, lactic acid, malic acid, phosphoric acid, and tartaric acid.

18. The process of claim 16, wherein the acid is citric acid.

19. The process of claim 14, wherein the saccharide comprises dextrose.

20. The process of claim 14, wherein the reaction mixture comprises dextrose in an amount from about 30% to about 50% by total saccharide weight.

21. The process of claim 14, wherein the amount of saccharide having a DP ranging from 1 to 4 is present in the reaction mixture in an amount of from about 30% to about 50% by total saccharide weight.

22. The process of claim 22, wherein the amount of saccharide having a DP of 200 or greater is present in the reaction mixture in an amount of from about 30% to about 50% by total saccharide weight.

23. The process of claim 14, said process yielding a mixture of saccharide-derivatized oligosaccharides having an Mₙ of at least 5000 g/mol.

24. The process of claim 14, said process yielding a mixture of saccharide-derivatized oligosaccharides having an Mₙ of at least 10000 g/mol.

25. The process of claim 14, said process yielding a mixture of saccharide-derivatized oligosaccharides having a DE ranging from 15 to 40.
